(19) United States
(12) Patent Application Publication
(10) Pub. No.: US 2008/0294130 A1
(43) Pub. Date: Nov. 27, 2008

(54) Device and Technique for Semen Collection from Boars

(76) Inventor: Christian Simmet, Tiefenbach (DE)

Correspondence Address: Matthew K Ryan;Frommer Lawrence & Haug, 745 Fifth Avenue, New York, NY, 10151, US

(21) Appl. No.: 11/658,211

(22) PCT Filed: Nov. 7, 2005
(86) PCT No.: PCT/EP05/11886

§ 371 Date: Jan. 23, 2007

(30) Foreign Application Priority Data
Nov. 8, 2004 (DE) 10 2004 053 796.8

(51) Int. Cl. A61F 5/451; A61B 17/43

(52) U.S. Cl. 604/349; 600/33

(57) ABSTRACT

Device for semen collection from boars consisting of a grip strip and a clamping fixture for a detachable fixing of the grip strip placed around the boar penis in a way that the necessary pressure for the ejaculation is exerted by the fixed grip strip.

The following invention relates to a device for semen collection from boars.

Usually the semen of boars is collected using a so-called collection dummy for boars which is accepted as a mounting partner and mounted by the sexually stimulated boar. The so-called hand method is normally applied during the process of semen collection. The person collecting the semen grasps the spiral-shaped tip of the boar's penis with one hand protected by a disposable glove as soon as the boar has started search movements and the penis tip has left the prepuce. Now, the semen collector tries to stimulate the penis tip located in his fist by applying a partly pulsating pressure and to achieve a complete erection. Once erection is attained, the ejaculation begins, and thus the actual semen collection. The ejaculate is collected in a cup.

Since the pressure exerted on the boar penis must be maintained during the entire ejaculation process in order to prevent, in particular, a premature abruption of the ejaculation and thus a loss of semen, the semen collecting technician must not withdraw from the dummy through the entire process of ejaculation and has to continuously exert the necessary relatively high pressure with his hand. Thus, the process of semen collection requires a relatively high expenditure of energy and is uncomfortable for the semen collecting technician.

To facilitate and automate the process of semen collection devices were suggested, such as an artificial vagina provided with an internal tube that can be filled with warm water and air, with which the necessary pressure and thermal stimulus exerted on the tip of the boar's penis during ejaculation can be generated. After having placed the erected boar penis in the artificial vagina and having inflated the internal tube, e.g. using a bellows, the ejaculation process can be finished, no longer requiring a person for holding.

The disadvantage of this artificial device for semen collection is, however, that a time-consuming sterilization of the artificial vagina is necessary after each process of semen collection in order to prevent hygienic problems. Furthermore, using the artificial vagina the high sensibility of a natural hand, especially required at the beginning of the ejaculation process, cannot be achieved. Consequently, with this method semen cannot be collected from all boars, which is a disadvantage for breeding farms.

That is the reason why the natural hand of a semen collecting technician is still used during most semen collections from boars.

It is the task of the following invention to provide an optimised device for semen collection from boars, with which almost complete ejaculates of good hygienic quality can be yielded from boars being able to mate, as well as to provide a corresponding procedure.

The solution to this task is a device for semen collection from boars with the characteristics according to Claim 1, as well as a procedure with the characteristics of Claim 25.

According to the invention, the device for semen collection from boars consists of a grip strip and a clamping fixture, with which the grip strip can be fixed, enabling a detachable clamping of the grip strip placed around the boar penis.

According to the invention, the device has the advantage that the grip strip can be placed into a hand with which the boar penis can be grasped in accordance with the usual procedure during a semen collection and can be led to erection and ejaculation while the sensibility of the hand of the semen collecting technician can be directly applied. After the beginning of the ejaculation the grip strip placed around the boar penis can be clamped in a clamping fixture without interrupting the pressure exertion so that the required pressure can now be exerted by the clamped grip strip, no longer requiring the semen collecting technician for holding or exerting any pressure. According to the invention, using this device, complete ejaculates of good hygienic quality can be yielded from all boars being able to mate.

According to the invention, the grip strip is preferably made of a flexible, compressible material in order to achieve a pressure coming close to the one exerted by a natural hand. Furthermore, regarding the grip strip a rough, non-slip surface is preferred so that the spiral-shaped tip of the boar penis can be securely fixed with the grip strip.

Suitable materials for the grip strip according to the invention are, in particular, low heat conducting materials which convey a sensation of heat on the skin. PUR, EPDM, silicone, latex rubber, microcellular rubber or other suitable, especially foamed materials are preferred.

Moreover, at least one surface of the grip strip can be provided with knobs, in particular, round or angular ones or of any other form and/or with longitudinal and horizontal stripes of the same or different height and width. The knobs or stripes are placed particularly on the side facing the boar penis in order to generate a surface as similar as possible to the natural situation, i.e. the natural cervix. The knobs and/or stripes are preferably arranged in a pattern. The knobs can be arranged in rows, for example, perpendicular to the long side of the grip strip, with the knobs of each row staggered to those in adjacent rows.

Furthermore, according to the invention, the grip strip can be provided with one or several adhesive sections on at least one surface so that it can be adhered to a hand and particularly to the surface of a hand glove, in particular a disposable hand glove. The grip strip can also be held to the palm of the hand using one or several holding straps placed over the back of the hand.

According to the invention, the grip strip preferably has a length between 10 and 30 cm, a maximum width between 2 and 10 cm and a thickness between 0.1 and 3 cm, which is particularly dependent on the material selected for the grip strip. The grip strip can also have the form of a closed ring.

Moreover, according to a preferred design, the grip strip has a form in which the width of the grip strip decreases towards the ends of the same. For instance, a strip divided into three sections of about the same length is conceivable, of which the middle section has a constant width and each of the two end sections has a width decreasing towards the ends.

According to another type of design, the grip strip can contain a heat dissipating material, which, for example, is embedded therein. Thus, the erection or the ejaculation process can be positively influenced and the result of the semen collection further optimised. Suitable heat dissipating materials are, for example, crystalline substances, in which mechanical stress triggers off a phase change leading to a heat release. Mechanical stress can be produced, for example, by exerting pressure or by bending a substance arranged in the form of a rod or a plate.

According to another design of the invention, the device includes a plane body which, surrounding the boar penis at least partially, can be arranged under or above the grip strip or be tightly connected to the grip strip and extends from the same. The plane body is preferably made of plastic, in particular of the same material the grip strip is made of. It is a sheet which can be bent into the form of a tube, with a sufficient width so that the boar penis can be surrounded at least partially and with a length so that the sheet extends from the grip strip placed around the boar penis beyond the penis tip. According to an especially preferred design, the sheet has the form of a cloth or an appendix which extends from a middle section of the grip strip perpendicular to its longitudinal direction, i.e. perpendicular to the connecting line between the ends, and is made of one piece with the grip strip.

According to the anatomy of the boar the urethra protrudes at the tip of the penis at an angle relative to the longitudinal extent of the boar penis so that the ejaculation jet is laterally oriented and relatively difficult to collect. The sheet, designated in accordance with the preferred type of design, has a form which at least partially surrounds the boar penis and, in particular, the penis tip so that the ejaculation jet released sideways by the boar penis hits the sheet and from there drops into a receptacle intended for the collection of the ejaculate. Thus, during semen collection, loss can be avoided and the yield of the semen collection process can be further increased.

According to another type of design, a bag or receptacle for collection of the ejaculation can be shaped or fixed at the grip strip. Preferably the bag extends essentially perpendicular to the grip strip and is closed at the end far from the grip strip. The bag can be connected to the grip strip by fusing or bonding over a part of its girth, i.e. over 180°, for instance, or, if applicable, be made of one piece with the grip strip. Preferably the bag is made of plastic, e.g. polyethylene, polypropylene or also of the same material as the grip strip and has a length of 20 cm and a diameter of 5 cm, for example. It serves preferably as a collection receptacle for the normally hygienically impure ejaculate or pre-sperm fraction, ejaculated at the beginning of the semen collection process and normally hygienically impure so that it can be separated from the hygienically purer ejaculate used for breeding purposes. The bag can be removed from the grip strip after a sufficient amount of pre-sperm ejaculate has been collected, whereas the ejaculate being released during the further ejaculation process is collected in another receptacle. The bag is perforated, preferably along its girth next to the grip strip, to facilitate the detachment.

According to another type of design, a tubular body, which is preferably also fixed at the grip strip, is placed over the above mentioned bag. However, the end of this body most distant to the grip strip is open. After the bag connected to the grip strip has been removed, the tubular body serves to collect the hygienically pure ejaculate and to drain it off into a receptacle. Since the tubular body is not contaminated with the pre-sperm fraction collected in the bag and, as the case may be, could even be sterile depending on the manufacturing process and storage, it can be brought into contact with the hygienically pure ejaculate while fulfilling a similar function as the above mentioned appendix or plane body fixed at the grip strip and designed to drain off the ejaculate. Regarding the material and fastening of the tubular body at the grip strip, the same applies as mentioned above regarding the bag connected to the grip strip.

The bag and the tubular body surrounding it are preferably made of one piece. Alternatively, it is also conceivable to use the bag connected to the grip strip combined with the above mentioned appendix or plane body, or also a grip strip only combined with a tubular body. Instead of a closed bag it would also be conceivable to arrange two tubular bodies on top of each other and fix them at the grip strip. In this case the pre-sperm fraction could drip off or be collected by means of a receptacle arranged at the end of the internal tubular body. Also in this case the internal tubular body would preferably be removed from the grip strip after termination of the pre-sperm fraction phase, in order to conduct the sperm-rich and hygienically clean part of the ejaculate without contamination into the semen collection receptacle by means of the remaining external tubular body.

Furthermore, in order to stimulate the boar penis, a conceivable feature would be, for example, a tube-shaped device, placed in or on the grip strip, which perhaps intermittently, could be filled with, for example, a gas or a liquid, perhaps heated, or be loaded with pressure.

According to the invention, the clamping fixture, in accordance with a preferred type of design, has been developed to embrace two fingers extending parallel to one another, between which the ends of the grip strip can be placed and clamped, overlapping one another. It would also be conceivable to fix or hook one end of the grip strip into the clamping fixture and to clamp only the other end.

According to the invention, the clamping fixture, in accordance with another preferred type of design, has at least one swiveling and lockable clamping jaw which enables detachable clamping of the end/s of the grip strip. In this regard, a clamping jaw is preferred which can be swiveled around an axis parallel to one of the fingers.

According to a preferred type of design, at least one of the fingers has a rotatable roller which can preferably be swiveled around an axis parallel to the long side of the finger. Preferably, the roller is freely rotatable in one direction, while the rotation in the other direction is selectively lockable or releasable with the help of a construction feature. Moreover, both fingers can be developed as rotatable parallel rollers so that the ends of the grip strip can be tightened between them and remain clamped due to friction between the rollers and the grip strip when the rotation of the rollers is blocked in the other direction.

According to another especially preferred type of design, the clamping fixture is provided with at least one, if necessary two tight rollers with increased rotary resistance suitable for fixing the grip strip. The rotary resistance is selected so as to ensure a sufficient fixation of the grip strip. With this kind of clamping fixture there is no need for a feature to lock or release the roller(s). The grip strip is simply loosened by turning the roller(s) with the fingers in the opposite direction to the pressure exerted on the boar penis. A clamping fixture of this kind has proved to be very practical because it is easy to handle, and it provides sufficient fixation strength.

Preferably one or both of the rollers can be connected to a drive with which traction can be exerted on the grip strip by turning the rollers, and thus a pressure on the boar penis.

According to the invention the clamping fixture can be developed in such a way that a permanent or intermittent pressure can be exerted on the penis, or traction on the grip strip, and thus on the penis tip, during the process of semen collection. This is especially possible with a clamping fixture which, as indicated above, is provided with one or two rollers and one drive. Regarding this type of design the drive can particularly be operated in such a way that a permanent or intermittent pressure or traction is exerted.

Furthermore, the ejaculation process can be stimulated in the preferred manner by pressing movements, tapping movements with variable length of the pressing phase, or vibrations, which are exerted on the clamping fixture or the grip strip or the penis itself by means of a suitable additional fixture, e.g. a vibrator, if applicable.

In order to be able to attach the clamping fixture to a stationary fixture and in particular to a dummy, thus relieving the semen collecting technician, the clamping fixture is, according to another type of design, provided with a fixing unit for attachment to a stationary fixture. Thus, for the remaining process of ejaculation no further hand movements are required by the semen collection technician after he has fixed the clamping fixture, with the boar penis placed therein, at the dummy.

Moreover, according to the invention a technique for semen collection from boars is made available which includes the following steps: stimulation of the boar penis, placement of a grip strip in the palm of the hand, grasping a boar penis so that the penis is enclosed at least over half of its girth by the grip strip, placing the grip strip in a clamping fixture and fixing the grip strip in the clamping fixture after erection has been achieved so that the pressure necessary for the ejaculation can be exerted by the fixated grip strip.

According to the invention, using this technique a certain part of the semen collection from boars can be automated so that a permanent presence of a semen collecting person or semen collecting technician is no longer required, while the pressure force required for the ejaculation process is applied by the device for semen collection according to the invention. Furthermore, according to the invention the technique is economic since the grip strips are produced and can be used as disposables, and no sterilisation is required for each individual process of semen collection.

According to the invention, the procedure may include that, according to one type of design, a plane body partially surrounding the penis tip is placed around the boar penis. The plane body, preferably made of plastic, the same material the grip strip is made of or another semen-friendly material, and preferably bent into the form of a tube or a funnel, serves to collect the ejaculation jet released at an angle to the longitudinal extent of the boar penis and to conduct it into a receptacle. The receptacle preferably includes a semen filter.

According to a preferred type of design, the procedure may also include the separation of a receptacle or tube-shaped body connected to the grip strip after the beginning of the ejaculation process in order to isolate the pre-sperm fraction ejaculated in the initial phase of the ejaculation process. Thus, the hygienically impure ejaculate especially released at the beginning of the ejaculation process can be separated from the pure ejaculate intended for breeding purposes. After removing the bag or tube-shaped body, the ejaculate released thereafter is collected in a receptacle. If applicable, an external tube-shaped body also connected to the grip strip and placed above the receptacle before the separation of the bag or tube-shaped body may be conceivable in order to collect the released ejaculate and conduct it into the receptacle.

According to another type of design, the procedure also includes the step of attaching the clamping fixture to a stationary fixture, in particular a dummy, so that the fixture does not need to be held during the ejaculation process.

According to another type of design, the procedure also includes the step of stimulating the boar penis and/or the ejaculation using a hand and the grip strip placed in it, before placing the ends of the grip strip in the clamping fixture. Thus, it is possible to use the sensibility of the natural hand for the initiation of the ejaculation process, which cannot be achieved with an artificial device, e.g. an artificial vagina.

According to another type of design, the procedure may also include the step of stimulating the boar penis during the process of semen collection by permanent or intermittent pressure, pulling or pressing movements or vibrations which are exerted on the clamping fixture or the grip strip, or the penis itself.

Furthermore, the procedure preferably includes the step of adhering the grip strip to the inner surface of a hand glove at the beginning and/or holding it by means of one or several fastening straps.

Further details, advantages and improvements of the invention result from the subclaims and from the following description of a preferred example of type of design with reference to the diagrams. It is shown in:

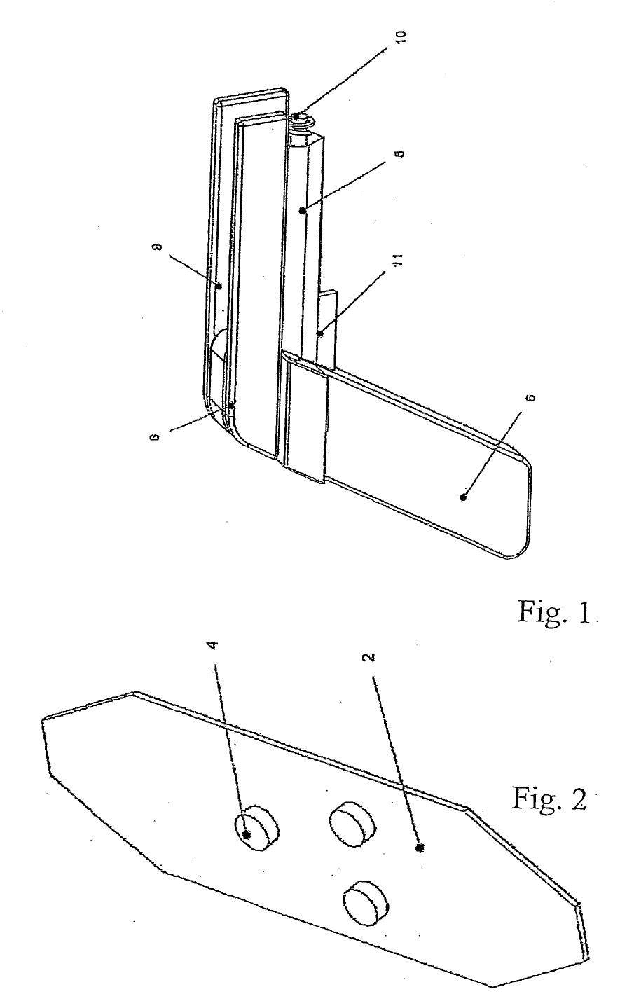

FIG. 1 a first type of design of a clamping fixture according to the invention;

FIG. 2 a grip strip according to a preferred type of design of the invention;

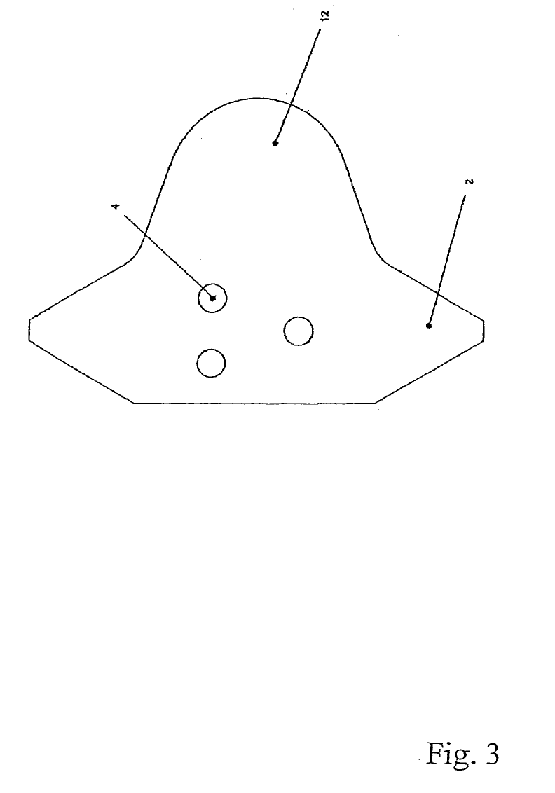

FIG. 3 a grip strip according to another preferred type of design of the invention;

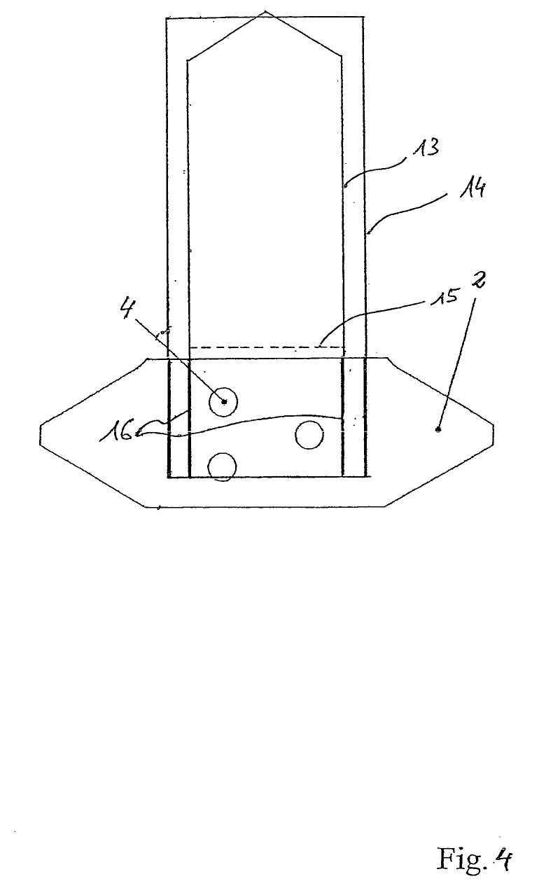

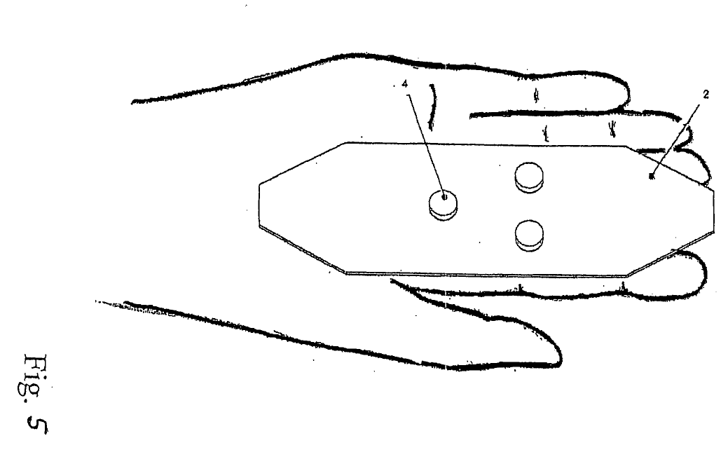

FIG. 4 a grip strip according to another preferred type of design of the invention;

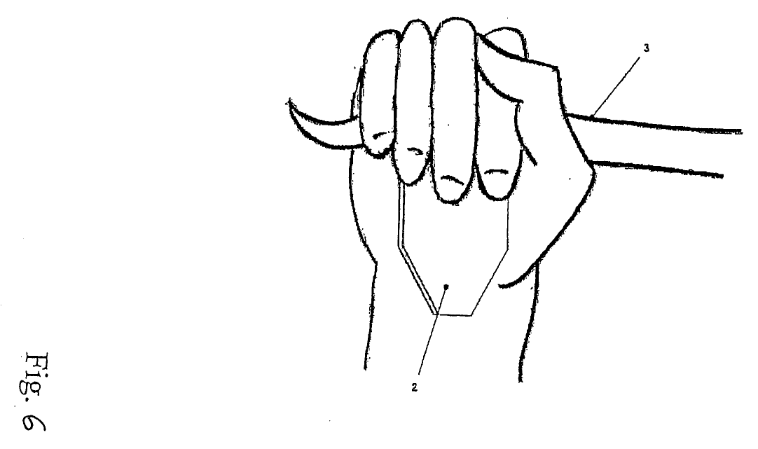

FIG. 5 a grip strip placed in the inner surface of a hand;

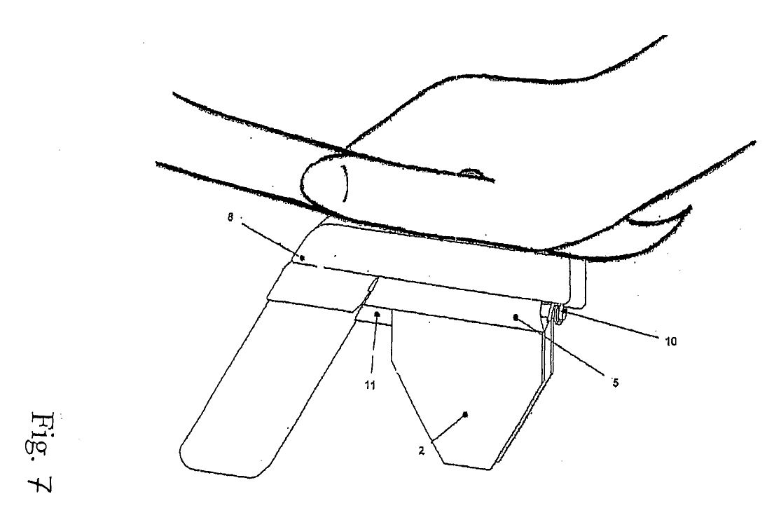

FIG. 6 the application of the grip strip to an erected boar penis;

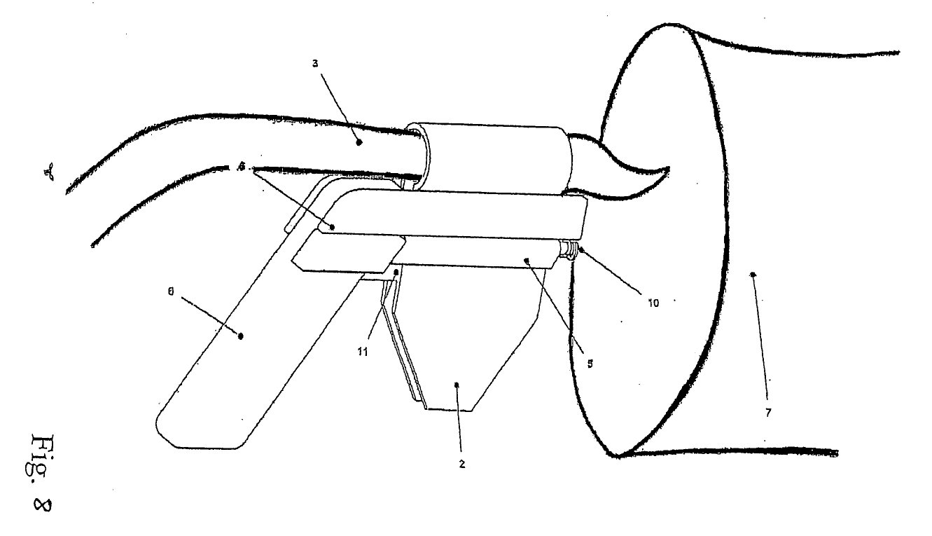

FIG. 7 the grip strip attached to a fixing unit while surrounding a boar penis;

FIG. 8 an illustration of the boar penis enclosed by the grip strip and attached to the fixing unit, and of a cup for the collection of the ejaculate.

A clamping fixture 1 according to the invention and in accordance with a special type of design, and grip strips 2 developed in accordance with three types of design, are especially illustrated in FIG. 1 to 4. The clamping fixture shown in FIG. 1 has two forklike fingers or pins 8, 9, parallel to one another at a short distance and connected at one end, between which the ends of the grip strip 2 shown in FIG. 2 to 4 can be passed through after having been placed around a boar penis 3. The fingers 8 and 9 have a rectangular cross section here, but can have any other cross section, e.g. a round or an angular one. The clamping fixture 1 includes, in addition, a clamping jaw 5 placed under one finger 8 and rotatable around an axis 10 essentially parallel to the finger 8, with which the ends of the grip strip can be clamped between the fingers 8, 9. In order to rotate the clamping jaw around the axis 10 the clamping jaw is provided with a nose 11 which can be turned to clamp the ends. Clamping jaw 5 is preferably stressed with a spring (not illustrated) with which the clamping jaw 5 can be held in the clamping position in which the ends of the grip strip 2 are clamped so that the grip strip 2 can be further tightened by traction at one or both ends of the grip strip 2 against the force of the spring. On the other hand, the clamping jaw 5 is constructed and placed on the clamping device 1 so as to be blocked against a tractive force in the opposite direction. Moreover, the clamping device 1 has a fixing unit 6 with which the clamping device can be fixed to a stationary device, a dummy, for example.

In contrast to the illustrated type of design, the clamping device may also have rotatable, parallel rollers placed on one or both fingers, with the roller preferably freely rotatable in one direction and the rotation in the other direction selectively blockable. Thus, the ends of the grip strip 2 can be fastened by exerting traction on them, e.g. by hand, so that they cannot come loose. Furthermore, alternatively one or, if necessary, two tight rollers may be conceivable, whose rotary resistance is selected so as to achieve, with it alone, a sufficient fixation of the grip strip. With this kind of clamping fixture there is no need for a feature to lock or release the roller(s). The grip strip is simply loosened by turning the roller(s) with the fingers in the opposite direction to the pressure exerted on the boar penis. It would also be conceivable to connect at least one roller with a drive, e.g. a mechanical drive or engine, with which the ends of the grip strip can be automatically fastened or loosened. By means of this drive an intermittent traction can also be exerted on the grip strip, or pressure on the penis, which contributes to additional stimulation of the penis and to ejaculation. Moreover, it is conceivable to exert with this drive or, if applicable, by means of another drive, or, if necessary, by means of an eccentrically driven feature, additional pressing movements or vibrations on the boar penis for its stimulation. In addition, a vibration fixture at the clamping fixture may also be conceivable.

The grip strip 2 is preferably made of a flexible, compressible material which preferably exhibits a rough and non-slip surface so that slipping on a boar penis 3 enclosed by the grip strip can be reduced or avoided. The grip strip 2 is preferably made of PUR, EPDM, silicone, latex rubber, microcellular rubber or another suitable material, i.e. especially a foamed material. Due to their thermal insulation properties these materials convey a sensation of heat on the skin, which can have a positive influence on the ejaculation. As can be seen in FIG. 2, the surface of the grip strip is provided with knobs 4 on one side. In principle, the knobs 4 can have any form, especially a round angular, elliptical one etc. Furthermore, it is preferred to arrange the knobs 4 in a certain pattern or arrangement, as it is shown in FIG. 2, for example. Instead of or together with the knobs 4 longitudinal and/or horizontal stripes (not illustrated) may also be conceivable, which may have different heights and/or widths, if necessary. With this special arrangement shown in FIG. 2, an anatomy, approximate to the natural cervix of a sow, is generated, with the help of which the process of semen collection can positively be influenced.

The grip strip preferably has a length between 10 and 30 cm, a maximum width between 2 and 10 cm and a thickness between 0.1 and 3 cm. Especially the thickness of the grip strip is dependent on the choice of the material. As can be seen in FIGS. 2 and 3, the grip strip has a reduced width towards its ends so that the ends can be clamped more easily between the fingers 8, 9 of the clamping fixture 1 and fixed there. The grip strip may also be conceivable in the form of a closed ring. The other surface of the grip strip 2, not illustrated in the figures, can be provided with adhesive strips with which it can be adhered to an area of a hand glove analogous to the palm of a hand, in particular a disposable hand glove. Furthermore, on the grip strip at least one fastening strap may be conceivable, in addition to or instead of the adhesive straps.

Moreover, the grip strip can contain a heat dissipating material, which, for example, is embedded inside in the form of padding. Suitable heat dissipating materials are, for example, certain crystals, in which mechanical stress, i.e. when the strip is bent, triggers off a phase change due to which heat is released.

On the inner or outer side of the grip strip 2, or inside, a tube may be conceivable, in addition, which is blown up with air to increase pressure, or, perhaps by means of a pump, is filled under pressure with a liquid, perhaps heated. Regarding the pressure, it can especially be an intermittent pressure whereby the ejaculation is further stimulated and the quantity of yielded ejaculate may be optimised.

A grip strip according to another preferred type of design is shown in FIG. 3. The grip strip illustrated there differs from the grip strip shown in FIG. 2 in that it has an appendix or sheet 12, which extends from about midway between the ends, perpendicular to the longitudinal direction, and preferably beyond the penis tip when the grip strip 2 is fixed. It serves to collect an ejaculation jet released sideways relative to the longitudinal extent of the boar penis so that this ejaculation jet drops from the appendix 12 into a receptacle placed underneath. This is an important aspect, especially during semen collection from boars because the urethra of the boar protrudes from the penis perpendicular to its longitudinal extent and thus, also the released ejaculation jet is not directed lengthwise to the penis but sideways. The appendix shown in FIG. 3 has the form of a nose or a Gaussian (normal distribution) curve. The extension of the appendix from the grip strip 2, in the shown example, corresponds approximately to the width of the grip strip. However, the size and the form of the appendix are variable and may be chosen according to the requirements. The width or extension of the appendix 12 is advantageously selected so as to extend beyond the penis tip when the grip strip is placed around the boar penis. The form or contour is also variable and also an angular or a sinusoidal one could be chosen. The appendix can be made of one piece with the grip strip or be fixed to it, e.g. by sewing, adhering etc.

In addition to the above mentioned appendix at the grip strip, a bag 13 may alternatively be conceivable, which is made of plastic, e.g. polyethylene, polypropylene or also of the same material as the grip strip, and, as it is shown in FIG. 4, which essentially extends perpendicular to the grip strip 2 and is closed at the end far from it. The open bag 13 at the grip strip 2 is connected to the grip strip 2 by fusing or bonding over a part of its girth, i.e. over 180°, for instance, with one edge of the grip strip 2, and also at the margins 16 extending beyond the knobs 4. However, the bag could also be made of one piece with the grip strip 2. The bag 13 serves to collect the ejaculate and especially the normally hygienically impure pre-sperm fraction ejaculated at the beginning and not suitable for further use. After a certain quantity of the ejaculate not suitable for further use has been collected in the bag 13, it is removed from the grip strip, whereas the ejaculate being released thereafter is collected in another receptacle. To facilitate the detachment, the receptacle 13 is provided with a perforation 15, preferably along its girth at the grip strip.

A tubular body or tube 14, which is fixed at the grip strip 2 in the same way, can be located over the above mentioned bag 13. However, the end of this body most distant to the grip strip 2 is open. After the inner bag 13 has been removed, the tube 14 serves to collect the hygienically pure ejaculate and to drain it off into a receptacle. The illustrated combination of an inner bag 13 and a tube 14 placed over it has the advantage that the hygienically impure pre-sperm fraction can be separated without contamination of the tube 14, which serves to collect and drain off the hygienically pure ejaculate. The inner bag 13 and the outer tube 14 are preferably made of one piece and, i.e. by means of a joint seam 16, for example, are conjointly fixed at the grip strip 2. Instead of the above mentioned grip strip 2 with appendix or sheet shown in FIG. 3, it may also be conceivable to use a grip strip, as it is shown in FIG. 2, together with a tube with an open end, or the grip strip provided with the appendix combined with the bag, to collect the pre-sperm fraction. The mentioned bag 13 could alternatively also have two open ends.

The technique for semen collection using the device according to the invention is described in FIG. 5 to 8. At the beginning of the process of semen collection, if necessary, after an initial stimulation of a boar penis 3, at first a grip strip 2 is placed on the hand provided with a hand glove, if necessary by means of adhesive strips or a loop at an area of the hand glove analogous to the palm of a hand, as it is shown in FIG. 3. Prior to the potentially prerequisite steps of initial stimulation and possible washing of the preputial area the grip strip can be fixed to the hand glove and be protected from contamination by means of a second hygienic glove. After completion of the stimulation and washing the hygienic glove is taken off. Afterwards, the erect boar penis 3 is enclosed by the grip strip 2, as it is shown in FIG. 6, thus stimulating an ejaculation by exerting pressure with the hand grasping the grip strip and the boar penis 3.

If applicable, a sheet made of plastic or another material suited for a contact with semen, and with a length so that it extends beyond the penis tip, is placed under the grip strip 2. By placing the grip strip around the penis the sheet is formed into a tube and surrounds the penis at least partially depending on its width and form. According to a relevant type, the sheet could also be fixed directly at the grip strip 2 or be integrated in it, as it is the case with the grip strip 2 shown in FIG. 3, for example. With this type of the grip strip 2, instead of a separate sheet the grip strip 2 already provided with the appendix 12 is used, thus facilitating the handling. Since the urethra of the boar, as it is explained above, generally protrudes from the penis perpendicular to its longitudinal extent, the released ejaculation jet is not directed lengthwise to the penis but sideways so that it hits the sheet or the appendix and from there drops into a receptacle held underneath. By means of this sheet loss can be avoided during semen collection.

After a complete erection has been achieved the ends of the grip strip 2, still placed around the boar penis 3 and held by the hand, are inserted between the fingers 8, 9 of the clamping fixture 1, while the grip strip 2 is pulled so tight with the other hand between the fingers 8, 9 of the clamping fixture 1 that a sufficient pressure, corresponding to the pressure exerted by the other hand, is exerted on the boar penis 3 (see FIG. 6). Afterwards, the ends of the grip strip 2 are fixed in the clamping fixture 1 by rotating a clamping jaw 5 located on the clamping fixture 1. The boar penis 3 fixed in the clamping fixture is especially illustrated in FIG. 7. By means of the designated fixing unit 6 on the clamping fixture 1, the clamping fixture 1 can be fixed at a boar dummy (not shown) so that the ejaculation process can be finished, no longer requiring a semen collecting technician for intervening or holding. The ejaculate is collected in a receptacle 7. The receptacle can be a cup or a bag preferably provided with a semen filter.

When using a grip strip (not shown in FIG. 5 to 8) provided with the above mentioned bag 13 or, in addition, with a tube 14 placed over it, the penis tip is grasped so that its front part is situated at the edge of the bag 13 or reaches a bit inside. After the beginning of the ejaculation and after the hygienically impure pre-sperm fraction has been collected, the receptacle 13 is separated from the grip strip. The pure ejaculate released subsequently is collected by means of the tube 14, not contaminated by the pre-sperm fraction, and is drained off into another receptacle.

The tube 14, which is not contaminated, is preferably passed through a conical ring (not shown) and folded back on the ring. The conical ring, which exhibits an external diameter corresponding to the opening of the receptacle, is then inserted into the receptacle so far until it is connected to the receptacle in the same way as a plug, and a tight connection between the tube 14 and the receptacle has been generated. Thus, it is achieved that ejaculate draining off inside the tube transfers completely into the receptacle. Moreover, this device provides a closed system as far as possible for a contamination-free collection of hygienically pure ejaculate, and a contamination of the ejaculate in the receptacle by water, urine, dust, hair, dirt, faeces, bacteria etc. can be avoided.

After termination of the ejaculation process the relaxed boar penis can be removed from the grip strip by the boar without any support from the semen collecting technician.

To remove the grip strip the clamping jaw 5 of the clamping fixture 1 is loosened by turning the designated nose 11 on it, and the grip strip is pulled out. If the clamping fixture is provided with the above mentioned roller(s) with increased rotary resistance, the grip strip is simply loosened by turning the roller(s) by hand.

The type of design of the device according to the invention, shown in the figures, can be modified in numerous ways, and neither the clamping fixture nor the shown grip strip is definitive for the illustrated special type of design. In principle, any fixture with which two ends of a grip strip can be fixed is suited for a clamping fixture. Also concerning the grip strip a multitude of modifications regarding the form, the dimensions and the material is conceivable.

It may be conceivable, for instance, to provide the grip strip, in addition, with an inflatable tube placed on it, with the help of which the pressure exerted on the boar penis 3 can be varied. It would also be conceivable, for instance, to fix one end of the grip strip 2 at or hook it into the clamping fixture and to clamp only the other end.

The grip strip according to the invention is preferably sterilised before use and developed as a disposable. It can be stored in sterile packaging.

The features of the invention disclosed in the claims can be of importance to the realisation of the invention, either alone or in any combination of them.

1. Device for semen collection from boars consisting of a grip strip (2) and a clamping fixture (1) for a detachable fixing of the grip strip (2) placed around the boar penis (3) in a way that the necessary pressure for the ejaculation is exerted by the fixed grip strip (2).

2. Device according to claim 1, characterized by a grip strip (2) consisting of a flexible, compressible material.

3. Device according to claim 1, characterized by a grip strip (2) exhibiting a rough, non-slip surface.

4. Device according to claim 1, characterized by a grip strip (2) consisting of PUR, EPDM, silicone, latex rubber, microcellular rubber or other, especially foamed materials.

5. Device according to claim 1, characterized by a grip strip (2) of which at least one surface is provided with knobs (4) and/or with longitudinal and horizontal stripes of the same or different height and width.

6. Device according to claim 1, characterized by knobs (4) and/or stripes arranged in a pattern.

7. Device according to claim 1, characterized by a grip strip (2) of which at least one surface is provided with one or several adhesive sections and/or one or several fastening straps.

8. Device according to claim 1, characterized by a grip strip (2) having a length between 10 and 30 cm, a maximum width between 2 and 10 cm and a thickness between 0.1 and 3 cm.

9. Device according to claim 1, characterized by a grip strip (2) whose width decreases towards the ends.

10. Device according to claim 1, characterized by a grip strip (2) provided with a heat dissipating material.

11. Device according to claim 1, characterized by the device including a plane body arranged under or above the grip strip (2) or being tightly connected to the grip strip and extending from the same.

12. Device according to claim 11, characterized by a plane body being a sheet with a width so that the boar penis (3) can be at least partially surrounded by it, and a length so that the sheet extends from the grip strip placed around the boar penis to beyond the penis tip.

13. Device according to claim 1, characterized by including a receptacle (13) or bag connected to the grip strip (2).

14. Device according to claim 1, characterized by a tube-shape body (14), with open ends, connected to the grip strip (2).

15. Device according to claim 14, characterized by a receptacle (13) placed inside the tube-shaped body (14) and made of one piece with the same.

16. Device according claim 1, characterized by a clamping fixture (1) including two fingers (8, 9) extending parallel to one another, between which the ends of the grip strip (2) are placed and clamped, overlapping one another.

17. Device according to claim 1, characterized by a clamping fixture (1) being provided with at least one swiveling and lockable clamping jaw (5) which enables detachable clamping of the ends of the grip strip (2).

18. Device according to claim 1, characterized by a clamping jaw (5) rotatable around an axis parallel to the fingers (8, 9).

19. Device according to claim 1, characterized by at least one of the fingers (8, 9) having a rotatable roller.

20. Device according to claim 19, characterized by a roller freely rotatable in one direction, while the rotation in the other direction is selectively lockable or releasable.

21. Device according to claim 19, characterized by a roller with increased rotary resistance suitable for fixing the grip strip.

22. Device according to claim 19, characterized by including a drive with which the roller can be operated or locked.

23. Device according to claim 1, characterized by a clamping fixture (1) provided with a fixing unit (6) for fixing to a stationary device, in particular a dummy.

24. Device according to claim 1, characterized by a feature with which a permanent or intermittent pressure, pulling and/or pressing movements or vibrations can be exerted on the grip strip (2) and/or the boar penis.

25. Technique for semen collection from boars including the following steps:
stimulating the boar penis (3),
placing a grip strip in the palm of a hand,
grasping the boar penis so that the penis is enclosed at least over half of its girth by the grip strip (2), and
fixing the grip strip (2) by means of a clamping fixture after erection has been achieved so that the pressure necessary for the ejaculation is exerted by the fixed grip strip (2).

26. Technique according to claim 25, characterized by the placement around the boar penis (3) of a sheet-like body or appendix of the grip strip (2) at least partially surrounding the penis tip.

27. Technique according to claim 25, characterized by the semen ejaculated by the boar, being collected in a container (7), preferably in a receptacle or bag with a semen filter.

28. Technique according to claim 25, characterized by including the removal of a receptacle or tube-shaped body connected to the grip strip (2) after the beginning of the ejaculation process in order to isolate the semen or pre-sperm fraction ejaculated in the initial phase of the ejaculation process.

29. Technique according to claim 25, characterized by including the step of attaching the clamping fixture (1) to a stationary fixture, in particular a dummy.

30. Technique according to claim 25, characterized by including the step of stimulating the boar penis (3) or the ejaculation using a hand before placing the ends of the grip strip (2) in a clamping fixture (1).

31. Technique according to claim 25, characterized by a permanent or intermittent pressure, pulling, pressing movements, tapping movements with variable length of the pressing phase, and/or vibrations being exerted on the grip strip (2) or the boar penis (3) during the semen collection process.

32. Technique according to the claim 25, characterized by a grip strip (2) adhered to the inner surface of a hand glove and/or held by means of one or several fastening straps.
